(19) United States
(12) Patent Application Publication
(10) Pub. No.: US 2008/0286343 A1
(43) Pub. Date: Nov. 20, 2008

(54) Solid form

(76) Inventors: Dzenana Cengic, Brussels (BE); Olivia Darmuzey, Brussels (BE); Graeme MacLeod, Wezembeek Oppem (BE)

Correspondence Address: PATENT ADMINISTRATOR;FMC CORPORATION, 1735 MARKET STREET, PHILADELPHIA, PA, 19103, US

(21) Appl. No.: 11/803,996

(22) Filed: May 16, 2007

(51) Int. Cl. A61K 9/70; A61K 9/14; A61P 1/00; A61P 11/00; A61P 25/00; A61P 29/00; A61P 3/00; A61P 31/00; A61P 33/06; A61P 9/00

(52) U.S. Cl. 424/443; 424/484; 424/489; 426/282; 510/441

(57) ABSTRACT

A solid form comprising at least one film enrobing a compacted fill material having at least one active material contained in a matrix and having low friability, a density of at least 0.5 g/ml based on the total solid volume of the solid form and a tensile strength less than 0.9 MPa and which exhibits a controlled release profile for release of the active material. Zero order release may be achieved.

### FIELD OF THE INVENTION

This invention relates to a solid form comprising a film enrobing a matrix of a compacted fill material and a method of producing the solid form.

### BACKGROUND TO THE INVENTION

Active ingredients, for example pharmaceutical, agrochemical and detergent active ingredients may be delivered through a wide range of solid forms including tablets and capsules. Conventional tablets generally are highly compacted and have relatively high densities. In conventional tablets, the active ingredient is generally compacted with other components in a blend to provide the requisite structural integrity for the tablet. Delivery of the active ingredient in use may however be unsatisfactory due to the compaction level and it is known to add excipients to the formulation to aid disintegration or dissolution of the tablet to improve delivery, aid compaction, increase strength and increase robustness of the solid form. This may however impose constraints on the flexibility of the formulator in developing tablets containing the active ingredient.

Capsules generally include the active ingredient in a relatively non-compacted form. However, the lack of compaction together with the void space inherent within capsules mean that for a given large dose of active, the volume of the final solid form is greater than for more compacted solid forms. Increasing the size of the capsule to accommodate the required dose is undesirable for the user. Typically, capsules require a relatively high level of disintegrant to provide adequate disintegration of the solid form. Capsule shells may also be sensitive to moisture and present problems as regards storage and product shelf-life.

WO 03/096963 discloses solid forms and processes utilizing films to enrobe a fill material to a degree of compaction less than that generally used to make a tablet. It is specifically disclosed therein that because of the nature of the capsule produced that certain ancillary ingredients necessary in conventional tablet production may be omitted. It is further disclosed therein that, due to relatively loose compaction, components contained within a tablet which are “designed to disperse and breakup the tablet when it has reached the site of delivery, can be omitted, as the active ingredients in the capsule according to the present invention are in a non-compacted or at least less compacted form as compared to a conventional tablet, and this lesser compaction leads to the easy release and dispersal of active ingredients once the capsule film has dissolved, e.g., at the intended site of delivery”.

There remains a need to provide a solid form able to provide a suitable and reliable dose level, to provide effective delivery of the active ingredient, to tailor the release profile of the active in the solid form and to have satisfactory structural integrity. It is also desirable for the formulator to have flexibility in formulating the solid form.

### SUMMARY OF THE INVENTION

The present inventors have found that a solid form having a compacted fill material in the form of a matrix with a particular combination of characteristics in which the components are less compacted than in a tablet but more than in a capsule formulation provides beneficial delivery of the active ingredient at acceptable dose levels and with fewer or lower quantities of excipients typically employed in capsules or tablets.

The invention provides in a first aspect a solid form comprising at least one film enrobing a compacted fill material wherein:
i) said compacted fill material comprises at least one active material;
ii) said solid form shows a weight loss that is less than 1% during a 30 minutes USP friability test United States Pharmacopeia (USP) 29 Test Number 1216 (page 3046);
iii) said compacted fill material has a density of at least 0.5 g/ml based on the total solid volume of the solid form and a tensile strength less than 0.9 MPa;
iv) the compacted fill material comprises particles comprising the at least one of said active material contained within a matrix; and
v) the active material exhibits controlled release.

### DETAILED DESCRIPTION OF THE INVENTION

The term “matrix” is known in the art and is employed in its known sense and is taken to mean herein a continuum of the compacted fill material which contains discrete particles of active material wherein the continuum and discrete particles form a single monolithic entity. The matrix may have pores or capillaries through which components of the compacted fill material, for example the active material may pass in use so as to effect controlled release of the active material from the solid form.

The term “controlled release” as used herein refers to a solid form characterized by slower active release kinetics, compared to an immediate release solid form.

The term “immediate release” is employed herein in accordance with its meaning known in the art and refers to a solid form in which the active material is released rapidly after administration. A typical release rate for an “immediate release” active material in a solid form is suitably not less than 85% active material release in 60 minutes, preferably in 45 minutes and especially in 30 minutes in the test specified in United States Pharmacopeia (USP) Edition 29 Test Number 711 at page 2673 for said active material when said active material is placed in a dissolution medium as specified in the USP dissolution specification or selected from dissolution media specified in the USP according to the solubility properties of the active material. Suitably, where the active material is not a pharmaceutical active, water is used as the dissolution medium to determine whether the active in the solid form exhibits a controlled release.

This is referred to in the United States Pharmacopeia (USP) as “Q” time. The term “immediate release” includes “fast release”.

The solid form suitably comprises an active material which exhibits immediate release. The solid form may additionally comprise an active material which does not exhibit immediate release. If desired, the solid form may comprise an active material which exhibits immediate release and be free of an active material which does not exhibit immediate release. The solid form preferably comprises an active material exhibiting a fast release.

A typical release rate for a “controlled release” active material is suitably less than 85% active material release in 60 minutes, preferably less than 85% in 2 hours, especially less than 85% in 4 hours and desirably 85% in 6 or more hours.

The solid form comprises an active material exhibiting controlled release. Suitably the solid form comprises a further active material which does not exhibit controlled release. As desired, the solid form does not contain an active material which does not exhibit a controlled release.

Preferably, where the active material is a pharmaceutical active, at least one of said active material exhibits a controlled release in a United States Pharmacopeia (USP) dissolution test specified in USP Edition 29 Test Number 711 at page 2673 for said active material when said active material is placed in a dissolution medium as specified in the USP dissolution specification or selected from dissolution media specified in the USP according to the solubility properties of the active material. Suitably, where the active material is not a pharmaceutical active, water is used as the dissolution medium to determine whether the active in the solid form exhibits a controlled release.

Where a dissolution medium is specified in the USP for an active material, this is suitably employed in the dissolution test. Where there is either:
i) no USP test for the active material;
ii) more than one test for the active material; or
iii) the active does not meet the USP specification with the specified medium;
   the skilled person will select the most appropriate medium for the dissolution test from the USP dissolution media specified in the USP having regard to the dissolution characteristics of the active material and the desired release profile of the active from the solid form.

Examples of media in which the dissolution test may be carried out include: (i) the medium specified in the United States Pharmacopeia preferably for said at least one active material, (ii) water, (iii) 0.1 M HCl or (iv) phosphate buffer having a pH between 5.8 and 8.0.

Suitable active materials include a pharmaceutical active, food component or product, veterinary active, cosmetic component or product, an appetite suppressant, detergent component or product or nutraceutical component or product. Preferably, the solid form comprises at least one film enrobing a compacted fill material wherein the compacted fill material comprises at least one active material and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes USP friability test United States Pharmacopeia (USP) 29 Test Number 1216 (page 3046) and the compacted fill material comprises particles comprising the at least one of said active material contained within a matrix.

The inventors have further found that controlled release may be secured from the solid form with the compacted fill material having a low density as compared to known product forms which provide controlled release.

Where the active material is a pharmaceutical active, the objective of controlled release solid forms is to obtain a controlled, preferably constant, level of active in the plasma and to reduce the frequency of dose administration.

The film enrobing the compacted fill material is preferably a water-soluble film.

Desirably, the film is in intimate contact with the compacted fill material. By “intimate contact” it is meant that the film and the compacted fill material are in direct contact preferably over the entire internal surface of film although some areas not being in direct contact with the compacted fill material may be acceptable. For example, a tablet or other product form may be contained within the film or the film may have a lining or coating presenting a barrier between the compacted fill and the film.

Suitably the matrix of the compacted fill material comprises a polymer. The matrix may be soluble in aqueous medium such that in use the matrix swells and then dissolves whereby the active material is released. Alternatively, the matrix may be insoluble such that in use a solvent for example water enters the matrix and on reaching particles of the active, dissolves the active material in the dissolution medium.

In the case of a soluble matrix, the matrix is suitably a hydrophilic matrix and is preferably a water-swellable hydrophilic polymer. The matrix is suitably capable of swelling by gel formation, followed by erosion and dissolution in aqueous media.

Where an insoluble matrix is employed, it suitably comprises a hydrophobic polymer, for example a lipid. Suitably the active material is contained in the hydrophobic matrix which preferably remains intact during release of the active material. The matrix may comprise capillaries or may be porous. As desired, the matrix may comprise a soluble channeling agent distributed through the matrix which upon contact with aqueous solution leaches out of the matrix so leaving a porous matrix with tortuous capillaries. The active material may be released via dissolution and diffusion through the capillaries. The insoluble matrix may be inert to gastrointestinal fluids.

The release rate of the active material within the matrix may be tailored by controlling the porosity of capillary pathways in the matrix.

The present invention advantageously provides a solid form which is able to provide release of the active material in a controlled manner, desirably with zero order kinetics that is where the rate of release of the active material is independent of time.

Without wishing to be bound by any theory, it is believed that a soluble matrix may show two different mechanisms for release of the active material: firstly, hydration of the soluble polymer to form a gel layer which acts as a barrier both to further uptake of water and also to release of active material from the inner “dry” part of the solid form and so slows the release of the active material as compared to an immediate release profile. Secondly erosion of the gel layer to release further active from within the eroded gel layer is another mechanism of a controlled release. The relative contributions of swelling and dissolution on the one hand and erosion on the other provide the controlled release of the active material. When formation of the gel through hydration and erosion of it occur at the same rate, i.e. when the two mechanisms synchronize, the gel layer thickness is constant and zero-order release of the active material may be observed and non-linear release is obtained when this is not the case.

Where equilibrium between the matrix erosion and swelling/hydration is not achieved, zero order release is not achievable without the addition of less viscous polymers or “pore formers” so as to allow more rapid erosion.

Conventionally, desirable zero order release may be obtained using complex formulations in conventional technologies such as tablets or using sophisticated technologies such as RingCap®, Qtracoat®, Meltrex®, Geomatrix®, Smartrix®, Procise®. The present invention does not have the drawbacks associated with tablets and zero order release may be secured without the complexity and limitations of needing to employ proprietary technologies.

The solid form of the present invention enables the achievement of zero order release mechanism using simple formulations for the compacted fill of the solid form. Although the compacted fill density is relatively low in comparison with conventional solid forms such as tablets, it is higher than for the powder in hard capsules and sufficient to enable gel formation at an early stage of the solid form dissolution, while keeping the integrity of the solid form over a prolonged period of time.

Advantageously, the solid form of the present invention reduces some drawbacks of conventional solid forms, for example an initial burst of the active being released. A lower or no burst effect from the solid form of the present invention may advantageously be achieved and a wide range of polymers may be employed without the need to employ pore formers to achieve zero order controlled release due to the low density level of the compacted fill of the solid form.

As desired, the enrobed solid form of the present invention having an active material within a matrix provides a means of tailoring the release profile of the active avoiding the use of complex formulations.

Suitably the compacted fill material including the matrix is formed by a compaction step. The compaction process is preferably carried out at lower compaction force than conventionally applied in producing tablets. Varying the compression force provides a means of tailoring the porosity of the matrix so as to alter the release profile of the active material. Generally a more rigid and less porous matrix will release the active material more slowly than a less consolidated matrix.

Advantageously, lower compaction pressure in producing the solid form allows it to be formulated without a filler if desired. This has the further practical advantage of simplifying processing and reducing the time required to formulate the product.

A gel modifier or a channeling agent compound for example soluble salts, sugars or polyols, may be included in the matrix.

Preferably the compacted fill material has a density of less than 1.1 g/ml and more preferably less than 1.05 g/ml. The density of the compacted fill material is suitably at least 0.55 g/ml. Preferably, the density of the compacted fill material is from 0.55 to 1.04 g/ml, more preferably from 0.62 to 1.04 g/ml and desirably from 0.75 to 1 g/ml. The density of the solid form is suitably higher than that for conventional capsules and as the density contributes to the release profile of the solid form, this may be optimized by the formulator according to the release profile required.

The compacted fill material suitably has a tensile strength of less than 0.9 MPa, preferably less than 0.5 MPa, especially less than 0.2 MPa and particularly less than 0.1 MPa. The compacted fill has sufficient tensile strength to retain the physical integrity of the compacted fill material and is preferably at least 0.05 MPa. The robustness of the solid form is suitably provided by the enrobing film rather than by the compacted fill material.

The solid form of the present invention has excellent robustness or physical strength. The robustness of a solid form may suitably be defined by measuring the weight loss of 10 solid forms when rotated in a USP friability apparatus. This test is as set out in USP 29 <1216> p 3046. The solid form of the present invention shows a weight loss of less than 1% when tested for a 30 minutes in a friability drum. As conventional solid forms such as coated tablets are considered to be robust when the weight loss after 4 minutes of friability testing is less than 1% measured according to USP 29 <1216> p 3046, the solid form of the present invention is especially robust.

The density of the compacted fill material of the solid form of the present invention refers to the total weight of the fill material divided by the total volume of the solid form within the film material. This is typically referred to as the apparent density of solid forms. The apparent density of a conventional tablet is typically greater than 1 g/ml as disclosed in, Pharmaceutical Technology, 27 (4), 67-80. In a conventional hard capsule, the fill material is lightly tamped so as to form a very weak slug that breaks up in the capsule shell, due to the air space above it. In a conventional hard capsule, the density of the fill material is therefore similar to the bulk density of the loose powder. The latter is typically less than 0.5 g/ml as disclosed in, Pharmaceutical Technology, 27 (4), 67-80. The density of the compacted fill material of the present invention is at least 0.5 g/ml and preferably at least 0.55 g/ml based on the total solid form volume.

A typical method for determining the density D of the fill material in the present invention is to determine the fill weight W (1), the fill volume V, which depends on the size of the tooling used to manufacture the solid forms and to calculate D using equation (2).

(1) W=Wt−Wf(g), where Wt is the weight of the total enrobed solid form and Wf is the weight of the film enrobing the solid form.

D=W/V(g/ml) (2)

For a solid form of the present invention having a 70 microns thick film and made with oblong concave tooling of 16.6 mm length and 7.3 mm width, the volume V of the fill material is calculated using equation (3)
(3)V=(212.7+110.8t)/1000 (ml), where t is the sidewall thickness of the solid form (mm), typically measured using a micrometer.

For a tablet or compact that is made using 13 mm diameter flat round punches, the volume V of the fill material is calculated using equation (4)

(4)V=[π(13/2)²t]/1000 (ml), where t is the tablet thickness (mm), typically measured using a micrometer. Conventiontal tablets are considered robust when the tensile strength of the compacted fill material is at least 1.0 MPa for example as disclosed in Pharmaceutical Technology, p 52-62, April 2005 (Douglas McCormick, —Evolutions in Direct Compression). A typical method for determining the tensile strength for round flat faced cylinder shapes is to measure the crushing force (also called hardness) of compacts on a tablet hardness tester and calculate the tensile strength a using equation (5) Journal of pharmaceutical sciences, vol. 59 (5), 688-691 Determination of tablet strength by the diametral-compression test, (Fell J. T. and Newton J. M., 1970).
(5)σ=2P/πDt (MPa), where P is the crushing force (N), D is the compact diameter (mm), and t is the compact thickness (mm), typically measured using a micrometer.

Suitably, the said solid form has a tensile strength of at least 1.3 MPa

The present invention is also directed to a method of making the solid form of the present invention.

The invention in a further aspect provides for the use of a solid form according to the invention in a method of treatment of the human or animal body by therapy.

The invention also provides a solid form according to the invention for use in a method of treatment of the human or animal body by therapy.

A wide range of active materials having widely differing solubility characteristics may be employed in the present invention. The active material may have a solubility in water of 1 g in less than 1 g water, 1 g in 1 to 10 g water, 1 g active in 10 to 30 g water, 1 g active in 30 to 100 g water, 1 g active in 100 to 1,000 g water, 1 g active in 1,000 to 10,000 g water, and 1 g active in more than 10,000 g water.

Examples of suitable classes of pharmaceutical actives include an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, proton pump inhibitors and nasal decongestants.

Examples of suitable actives include paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, phenelzine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocainide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceutically or nutraceuticaly acceptable salts, acids, esters, isomers, and metabolites thereof.

Where more than one active material is present, the two or more actives may be from the same class or may be from different classes. Examples of combinations of active materials from different classes include an antibiotic in combination with one of a decongestant, an anti-inflammatory, a cough expectorant, a cough suppressant or an active for treatment of cough or common cold, a proton pump inhibitor, anti-hypertension and anti-cholesterol actives.

Examples of classes where two or more active materials from one class may suitably be employed include respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants, anti-HIV, anti-malaria actives.

Examples of particular combinations of active materials include: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 5-ASA and 4-APA acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

Low levels of active material, for example from 1 to 30% may be employed as desired. However, the compacted fill material preferably comprises a moderate to high amount of the active material and this will be selected according to the particular active material and the intended use of the product. By way of example, the compacted fill material suitably comprises active material at a level of 60 to 95%. A moderate level of active material is suitably at least 30% and suitably up to 75%. A high level of active material is suitably at least 75%, preferably at least 95%, for example 95 to 98% provided that a matrix may be formed in which the active material is contained.

The at least one active material may be in any form although in a preferred embodiment, the at least one active is a powder. The active material of the present invention is preferably a powder and this suitably includes such powders as granules, micronized powders, spray-dried powders, freeze-dried powders and pellets.

The compacted fill material may contain at least one material from which the matrix is formed, herein referred to as a “matrix former”. Examples of suitable hydrophilic matrix formers include hydroxypropylmethyl cellulose, sodium carboxymethyl cellulose, alginates, carrageenans, xanthan gum, locust bean gum, carbopol, guar gum, hydroxypropyl cellulose, methyl cellulose, polyethylene oxide, polymethacrylates, mannitol, polyvinyl alcohol.

The hydrophilic matrix former suitably has a viscosity in the range of 80-120,000 cPs. A 2% w/v aqueous solution of the matrix former at 20° C. is typically used to measure the viscosity.

Examples of a suitable insoluble matrix former include hydrogenated vegetable oils, microcrystalline wax and carnauba wax, ethylcellulose, polyamide, polyethylene, polyvinyl acetate, cetyl alcohol, glyceryl monostearate, glyceryl behenate, glyceryl monooleate, glyceryl palmitostearate, polacrilin potassium, stearic acid, stearyl alcohol, yellow wax, zein, hydrogenated castor oil.

The compacted fill may contain at least one filler. Examples of suitable fillers include excipients such as glidants, binders, pore formers and lubricants. Examples of suitable fillers include microcrystalline cellulose, dicalcium phosphate, lactose, calcium carbonate, calcium phosphate dibasic anhydrous, calcium phosphate dibasic dehydrate, calcium phosphate tribasic, powdered cellulose, silicified microcrystalline cellulose, cellulose acetate, compressible sugar, confectioners sugar, dextrin, dextrose, ethylcellulose, fructose, lactitol, starch, pregelatinized starch, sucrose, talc, xylitol, maltodextrin, magnesium carbonate, maltose, mannitol, polydextrose, sodium alginate, sodium chloride, sorbitol, sucrose, sugar spheres, acacia, carrageenan, carbomer, chitosan, hydroxypropylmethylcellulose, carboxymethylcellulose sodium, gelatin, guar gum, hydroxyethyl cellulose, hydroxypropyl cellulose, methylcellulose, povidone, zein, citric acid, sodium bicarbonate, alginic acid, carboxymethylcellulose calcium, colloidal silicon dioxide, low substituted hydroxypropyl cellulose.

When a filler is present, it may be present in an amount of less than 70% by weight of the compacted fill material, less than 60% by weight of the compacted fill material, less than 50% by weight, less than 40%, less than 30% by weight of the compacted fill material, from 1 to 25%, from 1 to 10% and suitably from 1 to 5% by weight of the compacted fill material. The enrobed solid form of the present invention may contain no filler (except where the filler is a matrix former) in the compacted fill material.

The film to be used to enrobe the present invention may be any film capable of enrobing the compacted fill material without adversely impacting the desired dissolution profile. The film to be used may comprise water soluble components, water insoluble components or may comprise soluble and insoluble components in combination.

Preferably, the compacted fill material is enrobed by a film comprising at least one water soluble polymer. Films generally useful in the present invention include those that are thermo formable and generally have dissolution rates appropriate for the preparation of rapid release, preferably immediate release, solid forms of the invention. Examples of such water soluble polymers include cellulosic materials such as hydroxyethyl cellulose, hydroxypropyl cellulose, hydroxypropyl methyl cellulose; polyvinyl alcohol; hydrocolloids such as carrageenan, alginate and pectin; and water soluble acrylates. Examples of water insoluble polymers include ethylcellulose, methacrylates and cellulose acetate. The films used in the invention may be gelatin free. The films may contain plasticizers such as lactic acid, citric acid, polyethylene glycol, sorbitol, glycerine, triethylcitrate, propylene glycol, phthalates, triglycerides, triacetin, tributylcitrate, etc. WO 2004/026284, WO 02/083779 and WO 03/095548 disclose further examples of films that may be used in the invention and such are incorporated herein by reference. Examples of films that may be used in the present invention are available under the trade name XGEL UNO from BioTec Films LLC, Tampa, Fla., US. Films for use in the present invention may be made in a conventional manner. If desired, an adhesive and use thereof can be used to aid in sealing the films together. Suitable adhesive compositions include those set forth in WO 04/10337 and WO 04/103338—both of which are incorporated herein by reference. The solid forms of the present invention may be enrobed and prepared in accordance with the methods disclosed in WO 03/096963, WO 05/030115, WO 05/030116 and PCT/GB2005/001077—all of which are incorporated herein by reference.

In a further aspect, the invention provides a method of making a solid form comprising at least one film enrobing a compacted fill comprising:
i) providing a first film shaped to define an interior volume for holding a compacted fill material and having an open end;
ii) depositing via the open end a fill material comprising an active material and a matrix former
iii) applying pressure to the fill material so as to compact the fill material;
iv) applying a second film over the said open end to close the said open end; and
v) sealing the first and second films together to enrobe the compacted fill material and provide the solid form.

The invention also provides for the use of a solid form according to the invention in which the active material comprises a pharmaceutical active for use in the manufacture of a medicament for treatment of the human or animal body by therapy.

A further aspect of the invention provides a solid form according to the invention in which the active material comprises a pharmaceutical active for use in a method of treatment of the human or animal body by therapy.

### DESCRIPTION OF THE DRAWINGS

The present invention is further described with reference to specific examples which are illustrative of the invention.

FIG. 1 shows the theophylline dissolution profile of Examples 1 and 2 and Comparative Example 3.

FIG. 2 shows the theophylline dissolution profile of Examples 1, 4, 5 and 6.

FIG. 3 shows the theophylline dissolution profile of Examples 2 and 7.

### EXAMPLES

### Materials

| Material | Grade | Supplier |
|---|---|---|
| Theophylline | USP grade | Shandong Xinhua |
| | | Pharmaceutical Co., Ltd. |
| benzyl alcohol | | EM Science |
| hypromellose (HPMC) | Methocel ® K4M | Dow Chemical |
| hypromellose (HPMC) | Methocel ® E 15LV | Dow Chemical |
| hypromellose (HPMC) | Methocel ® K15M | Dow Chemical |
| microcrystalline | Avicel ® PH 102 | FMC Corp, Philadelphia |
| cellulose | | PA |
| Triacetin | | Eastman |

### Methods

Fill material: The theohylline was sieved through a 24 mesh screen (710 microns) prior to weighing. Powders were weighed and blended for 15 to 20 minutes in a Turbula TF2 shaker mixer, a PharmaTech V-blender or in a Speedmixer DAC150FVZ-K for 5 seconds at 3000 rpm. The powder fill material was stored in a plastic bottle or double plastic bags until use.

Theophylline is slightly soluble in water (1 g in 100-1000 g water).

Tablets were prepared with a manual single punch press Specac with 13 mm flat punches. 500 mg powder was weighed and poured in the die and was compressed with a 15 kN force approximately.

Enrobed solid form: Soluble films known as XGEL UNO and supplied by Bio Tec Films LLC were cut into strips 6 centimeters by 20 centimeters approximately. The lower and upper films had a thickness of about 120 microns. The lower film was heated sufficiently to thermoform under vacuum into dose cups about 3 millimeters in height to conform to cavities (7.5 millimeters width by 16.75 length millimeters) with the cavity depth determined by height-adjustable dose-shaped lower pistons within the stainless steel die. The film strip was placed over the die and brought in contact with a heated TEFLON® coated surface by means of upward vacuum. The film was then drawn into the stainless steel die cavities by inverting the vacuum to form a strip of twelve thermoformed dose cups with 3.0 millimeters separation between adjacent dose cups. Some unused portion of the filmstrip was cut and removed. The fill composition was dosed (by volume) into the dose cups, though a paper funnel. Then the powder fill was lightly compacted in the dose cups with upper pistons, and the lower film was cut to separate the individual solid forms. The solid forms were then lifted by the lower pistons to expose a portion of the solid form sidewalls for application of the upper film to complete the enrobing of the solid form. An adhesive composed of 5% Methocel E15LV Premium, 45% Benzyl alcohol and 50% Triacetin, was applied (by transfer roller) to the upper filmstrip on the side to be pressed against the exterior sidewall of the dose cup. The upper film was placed over the solid forms containing the compressed powder fill and the film was heated by contact with the heating element using upper vacuum. The heated upper film was formed around the solid forms using the lower vacuum enclosing the fill material within the solid form by overlapping the upper film onto the sidewall of the solid forms. The top film was cut to separate the completed enrobed solid forms and the unused film was removed. The solid forms were further sealed by forcing them through a heated die under low pressure so that the cut film overlapping the sides was pressed smooth. All examples below used the apparatus set forth in WO 2005/030115,

Soluble HPMC containing films were used to enrobe the solid forms.

Dissolution was according to United States Pharmacopeia USP 29 with dissolution apparatus 1, baskets, for the enrobed solid forms and according to United States Pharmacopeia USP 24 with dissolution apparatus 2, paddles and sinkers for tablets. The solid forms were tested using 900 ml of simulated gastric fluid without enzymes according to USP 29 for 1 hour and 900 ml of simulated intestinal fluid without enzymes according to USP 29 thereafter.

### Examples 1 and 2 and Comparative Example 3

Solid forms of the present invention were composed of the following materials: 1) a blend made of 80% Theophylline and 20% Methocel K4M (medium viscosity HPMC grade for sustained release), and 2) a blend of 80% Theophylline and 20% Methocel K15M (high viscosity HPMC grade for sustained release). The tablets 3) were prepared for comparative purposes and were made of a blend of 60% Theophylline, 20% Methocel K4M and 20% Avicel PH102 (used to aid compaction)

The solid forms were compacted at approximately 15 kN.

Table I shows the mean weights of the dosage forms and their components (the fill materials), the Theophylline release in the dissolution test.

Despite the very low fill density, the release of Theophylline from the enrobed dosage forms of the present invention was prolonged, reaching a full release in approximately 24 hours of dissolution. The release of Theophylline from the enrobed dosage forms made with the lower viscosity grade of HPMC was faster after two hours of dissolution than the enrobed dosage forms made with the higher viscosity grade of HPMC. The active release profile was linear for both enrobed dosage forms of the invention, hence defining a zero order type of release. The comparative tablets showed a release profile that is non-linear, proportional to the square root of time, typical of tableted HPMC. Another typical characteristic of active release from HPMC tablets is the initial burst release and the subsequent decrease in release rate, which is shown by the tablets here and avoided using the enrobed solid dosage form of the present invention.

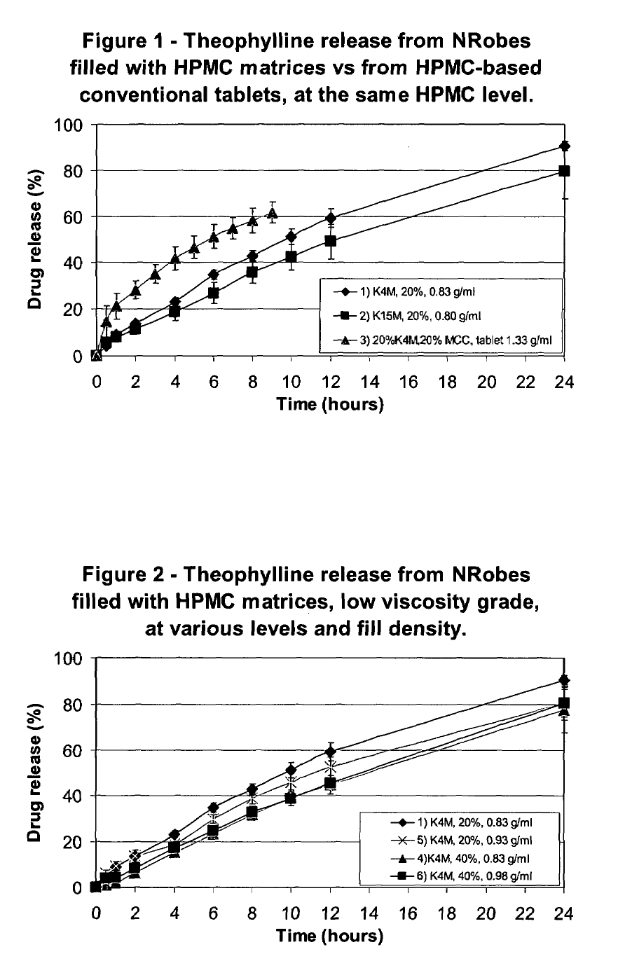

The active release curves of Examples 1 and 2 and Comparative Example 3 are shown in FIG. 1.

Examples 1 and 2 are within the scope of the invention. Example 3 is a comparative example.

TABLE I
Theophylline release from Matrix enrobed dosage forms of the present
invention

| | | | Comparative |
|---|---|---|---|
| Dosage Form | Example 1 | Example 2 | Example 3 (tablet) |
| Active | Theophylline | Theophylline | Theophylline |
| Total Active loading (%) | 80 | 80 | 60 |
| Fill Material (Matrix | 20% HPMC K4M | 20% HPMC K15M | 20% HPMC K4M + |
| former) | | | 20% MCC (filler) |
| Solid form weight | 448 | 431 | 501 |
| (milligrams) | | | |
| Fill weight (milligrams) | 408 | 391 | 501 |
| Fill Density | 0.83 | 0.80 | 1.33 |
| (grams/milliliter) | | | |
| Active release (%) after | 4 +/− 1 | 6 +/− 1 | 15 +/− 1 |
| 30 minutes | | | |
| Active release (%) after | 9 +/− 1 | 8 +/− 1 | 21 +/ 1 |
| 1 hour | | | |
| Active release (%) after | 35 +/− 2 | 27 +/− 5 | 51 +/− 3 |
| 6 hours | | | |
| Active release (%) after | 59 +/− 4 | 49 +/− 7 | Nm |
| 12 hours | | | |
| Active release (%) after | 91 +/− 2 | 80 +/− 12 | Nm |
| 24 hours | | | |
| Equation of trendline | y = 3.8298x + | y = 3.3118x + | y = −0.5922x2 + |
| from 1 hour through 24 | 7.0272 | 5.3879 | 11.26x + 6.3934 |
| hours data points | linear | linear | non linear |
| R² value of trendline | 0.96 | 0.98 | 0.98 |

### Examples 4, 5 and 6

The solid forms were composed of the following materials: 4) and 6) a blend made of 60% Theophylline and 40% Methocel K4M, and 5) a blend of 80% Theophylline and 20% Methocel K4M.

Table II shows the mean weights of the solid forms and their components (the compacted fill material) and the rate of Theophylline release in the dissolution test.

The active release rate was decreased with an increase in the HPMC content. The difference in fill density did not generate significant difference in release rate of the Theophylline at high HPMC content but an increase of fill density generated slower release at lower HPMC content. The active release profile was linear for all enrobed dosage forms of the invention, hence defining a zero order type of release.

The active release curves of Examples 1, 4, 5 and 6 are shown in FIG. 2.

TABLE II
Theophylline release from Matrix enrobed dosage forms of the present
invention

| Dosage Form | Example 1.1 | Example 4 | Example 5 | Example 6 |
|---|---|---|---|---|
| Drug | Theophylline | Theophylline | Theophylline | Theophylline |
| Total Active | 80 | 60 | 80 | 60 |
| loading (%) | | | | |
| Fill Material | 20% HPMC | 40% HPMC | 20% HPMC | 40% HPMC |
| | K4M | K4M | K4M | K4M |
| Solid form weight | 448 | 449 | 494 | 519 |
| (milligrams) | | | | |
| Fill weight | 408 | 409 | 454 | 479 |
| (milligrams) | | | | |
| Fill Density | 0.83 | 0.83 | 0.93 | 0.98 |
| (grams/milliliter) | | | | |
| Active release (%) | 4 +/− 1 | 1 +/− 1 | 6 +/− 2 | 4 +/− 1 |
| after 30 minutes | | | | |
| Active release (%) | 9 +/− 1 | 2 +/− 2 | 9 +/− 2 | 5 +/− 1 |
| after 1 hour | | | | |
| Active release (%) | 35 +/− 2 | 23 +/− 1 | 30 +/− 2 | 25 +/− 2 |
| after 6 hours | | | | |
| Active release (%) | 59 +/− 4 | 45 +/− 4 | 52 +/− 5 | 46 +/− 3 |
| after 12 hours | | | | |
| Active release (%) | 91 +/− 2 | 77 +/− 10 | 81 +/− 7 | 81 +/− 6 |
| after 24 hours | | | | |
| Equation of | y = 3.8298x + | y = 3.3595x + | y = 3.3532x + | y = 3.3748x + |
| trendline from 1 | 7.0272 | 1.4352 | 6.967 | 2.9005 |
| hour through 24 | linear | linear | linear | linear |
| hours data points | | | | |
| R² value of | 0.96 | 0.99 | 0.97 | 0.99 |
| trendline | | | | |

### Example 7

The solid forms were composed of the following materials: 7) a blend made of 60% Theophylline and 40% Methocel K15M.

Table III shows the mean weights of the dosage forms and their components (the fill materials), the Theophylline release in the dissolution test.

The active release rate was decreased with increase in the HPMC content. The active release profile was linear for both enrobed dosage forms of the invention, hence defining a zero order type of release.

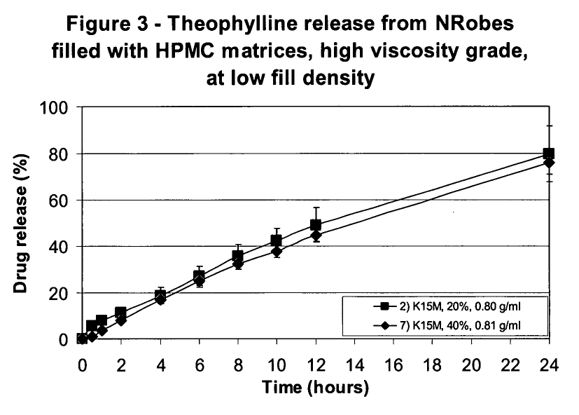

The active release curves of Examples 2 and 7 are shown in FIG. 3.

TABLE III
Theophylline release from Matrix enrobed dosage
forms of the present invention

| Dosage Form | Example 2 | Example 7 |
|---|---|---|
| Active | Theophylline | Theophylline |
| Total Active loading (%) | 80 | 60 |
| Fill Material | 20% HPMC K15M | 40% HPMC |
| | | K15M |
| Solid form weight (milligrams) | 431 | 436 |
| Fill weight (milligrams) | 391 | 396 |
| Fill Density (grams/milliliter) | 0.80 | 0.81 |
| Active release (%) after 30 | 6 +/− 1 | 1 +/− 0 |
| minutes | | |
| Active release (%) after 1 hour | 8 +/− 1 | 4 +/− 0 |
| Active release (%) after 6 hours | 27 +/− 5 | 25 +/− 2 |
| Active release (%) after 12 | 49 +/− 7 | 45 +/− 3 |
| hours | | |
| Active release (%) after 24 | 80 +/− 12 | 76 +/− 5 |
| hours | | |
| Equation of trendline from 1 | y = 3.3118x + | y = 3.2465x + |
| hour through 24 hours data | 5.3879 | 2.6489 |
| points | linear | linear |
| R² value of trendline | 0.98 | 0.98 |

1. A solid form comprising at least one film enrobing a compacted fill material wherein:
i) said compacted fill material comprises at least one active material;
ii) said solid form shows a weight loss that is less than 1% during a 30 minutes USP friability test United States Pharmacopeia (USP) 29 Test Number 1216 (page 3046);
iii) said compacted fill material has a density of at least 0.5 g/ml based on the total solid volume of the solid form and a tensile strength less than 0.9 MPa;
iv) the compacted fill material comprises particles comprising the at least one of said active material contained within a matrix; and
v) the said active material exhibits a controlled release.

2. A solid form at least one film enrobing a compacted fill material wherein the compacted fill material comprises at least one active material and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes USP friability test United States Pharmacopeia (USP) 29 Test Number 1216 (page 3046) and the compacted fill material comprises particles comprising the at least one of said active material contained within a matrix.

3. A solid form according to claim 1 or claim 2 wherein at least one of said active material exhibits a controlled release in a United States Pharmacopeia (USP) dissolution test specified in USP Edition 29 Test Number 711 at page 2673 for said active material when, said active material is placed in a dissolution medium as specified in the USP dissolution specification or selected from dissolution media specified in the USP according to the solubility properties of the active material.

4. A solid form according to claim 1 or claim 2 wherein the active material is in a form selected from granules, micronized powders, spray-dried powders, freeze-dried powders and pellets.

5. A solid form according to claim 1 or claim 2, wherein said active material comprises at least one of an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, proton pump inhibitors and nasal decongestants

6. A solid form according to claim 1 or claim 2, comprising at least two active materials wherein the active materials are selected from:
i) an antibiotic in combination with a decongestant, an anti-inflammatory, a cough expectorant, a cough suppressant or an active for treatment of cough or common cold, a proton pump inhibitor;
ii) an anti-HIV, anti-malaria active material, an anti-hypertension and anti-cholesterol,
iii) two or more active materials from the same class of active materials, the class being selected from respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants.

7. A solid form according to any claim 1 or claim 2 wherein the said at least one active material comprises paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, phenelzine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocainide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceuticaly or nutraceuticaly acceptable salts, acids, esters, isomers, and metabolites thereof.

8. A solid form according to claim 1 or claim 2, comprising at least two active materials wherein the active materials are selected from: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 5-aminosalicylic acid, 4-aminophthalic acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

9. A solid form according to claim 1 or claim 2 wherein said at least one active material is present in an amount of at least 30% by weight of the compacted fill material.

10. A solid form according to claim 1 or claim 2 wherein said at least one active material is present in an amount of at least 75% by weight of the compacted fill material.

11. A solid form according to claim 1 or claim 2 wherein said at least one active material is present in an amount of at least 95% by weight of the compacted fill material.

12. A solid form according to claim 1 or claim 2 wherein the at least one active material is present in an amount greater than 100 mg.

13. A solid form according to claim 1 or claim 2 wherein the at least one active material is present in an amount less than 100 mg.

14. A solid form according to claim 1 or claim 2 wherein the tensile strength of the compacted fill material is less than 0.2 MPa.

15. A solid form according to claim 1 or claim 2 in which the matrix comprises a hydrophilic matrix former.

16. A solid form according to claim 15, wherein said hydrophilic matrix former comprises at least one of hydroxypropylmethyl cellulose, sodium carboxymethyl cellulose, alginates, carrageenans, xanthan gum, locust bean gum, carbopol, guar gum, hydroxypropyl cellulose, methyl cellulose, polyethylene oxide, polymethacrylates, mannitol, polyvinyl alcohol.

17. A solid form according to claim 15, wherein said hydrophilic matrix former has a viscosity in the range of 80-120000 cPs.

18. A solid form according to claim 1 or claim 2 n which the matrix comprises an insoluble matrix former.

19. A solid form according to claim 18 wherein said insoluble matrix former comprises at least one of hydrogenated vegetable oils, microcrystalline wax and carnauba wax, ethylcellulose, polyamide, polyethylene, polyvinyl acetate, dibasic calcium phosphate, cetyl alcohol, glyceryl monostearate, glyceryl behenate, glyceryl monooleate, glyceryl palmitostearate, polacrilin potassium, stearic acid, stearyl alcohol, yellow wax, zein, hydrogenated castor oil.

20. A solid form according to claim 15 wherein the said matrix former is present in an amount less than 70% by weight of the compacted fill material.

21. A solid form according to claim 1 or claim 2 further comprising a filler.

22. A solid form according to claim 1 or claim 2, wherein said filler comprises at least one of microcrystalline cellulose, dicalcium phosphate, lactose calcium carbonate, calcium phosphate dibasic anhydrous, calcium phosphate dibasic dehydrate, calcium phosphate tribasic, powdered cellulose, silicified microcrystalline cellulose, cellulose acetate, compressible sugar, confectioners sugar, dextrin, dextrose, ethylcellulose, fructose, lactitol, starch, pregelatinized starch, sucrose, talc, xylitol, maltodextrin, magnesium carbonate, maltose, mannitol, polydextrose, sodium alginate, sodium chloride, sorbitol, sucrose, sugar spheres, acacia, carrageenan, carbomer, chitosan, hydroxypropylmethylcellulose, carboxymethylcellulose sodium, gelatin, guar gum, hydroxyethyl cellulose, hydroxypropyl cellulose, methylcellulose, povidone, zein, citric acid, sodium bicarbonate, alginic acid, carboxymethylcellulose calcium, colloidal silicon dioxide, low substituted hydroxypropyl cellulose.

23. A solid form according to claim 22, wherein said filler is present in an amount less than 70% by weight of the compacted fill material

24. A solid form according to claim 1 or claim 2, wherein said active material shows zero order release kinetics.

25. A solid form according to claim 1 or claim 2, wherein said active material shows first order release kinetics.

26. A solid form according to claim 1 or claim 2, wherein said active material shows a release, which is proportional to the square root of time.

27. A solid form according to claim 1 or claim 2, wherein the film enrobing the compacted fill material is a water-soluble film.

28. A solid form according to claim 1 or claim 2 in which the active material comprises a pharmaceutical active for use in a method of treatment of the human or animal body by therapy.

29. A method of treatment of the human or animal body by administering a solid form according to claim 1 or claim 2 in which the active material comprises a pharmaceutical to the human or animal body.

30. A method of making a solid form comprising at least one film enrobing a compacted fill comprising:
i) providing a first film shaped to define an interior volume for holding a compacted fill material and having an open end;
ii) depositing via the open end a fill material comprising the active material and a matrix former;
iii) applying pressure to the fill material so as to compact the fill material,
iv) applying a second film over the said open end to close the said open end; and
v) sealing the first and second water soluble film together to enrobe the compacted fill material and provide the solid form.
